(19) United States
(12) Patent Application Publication
(10) Pub. No.: US 2005/0148492 A1
(43) Pub. Date: Jul. 7, 2005

(54) Fragrance compounds

(76) Inventors: Jamie Brian Mankee, Sittingbourne (GB); David Munro, Kent (GB); Karl Andrew Dean Swift, Kent (GB); Kim Joyce Yarwood, Kent (GB)

Correspondence Address: MORGAN LEWIS & BOCKIUS LLP, 1111 PENNSYLVANIA AVENUE NW, WASHINGTON, DC, 20004, US

(21) Appl. No.: 10/470,101

(22) PCT Filed: Jan. 17, 2002
(86) PCT No.: PCT/GB02/00221

(30) Foreign Application Priority Data
Jan. 26, 2001 (EP) 01300714.1

(51) Int. Cl. A61K 7/46

(52) U.S. Cl. 512/1

(57) ABSTRACT

Novel compounds having the structure (I) where R and R¹ is each independently hydrogen or an alkyl, alkenyl, cycloalkyl or cycloalkenyl group having from 1 to 5 carbon atoms can have desirable odour properties, typically of a peach, fruity, lactone or nitrile character and find use in perfumes and perfumed products. Fragrance uses of 6-phenylhexan-2-one are also disclosed.

### FIELD OF THE INVENTION

This invention concerns novel fragrance compounds and methods of making them, and perfumes and perfumed products comprising the novel compounds.

### SUMMARY OF THE INVENTION

In one aspect of the present invention provides a compound having the structure 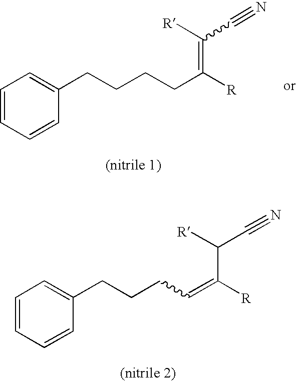
where R and R¹ is each independently hydrogen or an alkyl, alkenyl, cycloalkyl or cycloalkenyl group having from 1 to 5 carbon atoms.

For brevity and simplicity, such materials will be referred to as “the nitrile”, “the novel nitrile” or “the nitrile of the invention”.

The nitriles of the invention can occur in two different versions, dependent on the position of the double bond, which are referred to herein as version 1 or nitrile 1 and version 2 or nitrile 2, as indicated above. The invention covers each version alone and also mixtures of the two versions.

The nitriles of the invention can possess fragrance or odour properties which are generally regarded as interesting, pleasant or attractive, typically having peach, fruity, lactone or nitrile odour properties.

The odour properties of the nitriles of the invention mean that a nitrile or mixture of nitriles in accordance with the invention may be used as such to impart, strengthen or improve the odour of a wide variety of products, or may be used as a component of a perfume (or fragrance composition) to contribute its odour character to the overall odour of such perfume. For the purposes of this invention a perfume is intended to mean a mixture of fragrance materials, if desired mixed with or dissolved in a suitable solvent or mixed with a solid substrate, which is used to impart a desired odour to the skin and/or product for which an agreeable odour is indispensable or desirable. Example of such products are: fabric washing powers, washing liquids, fabric softeners and other fabric care products; detergents and household cleaning, scouring and disinfection products; air fresheners, room sprays and pomanders; soaps, bath and shower gels, shampoos, hair conditioners and other personal cleansing products; cosmetics such as creams, ointments, toilet waters, preshave, aftershave, skin and other lotions, talcum powers, body deodorants and antiperspirants, etc. Nitriles in accordance with the invention can show good substantivity to cloth, both wet and dry, and hence have good potential for use in fabric treatment products.

Other fragrance materials which can be advantageously combined with one or more nitriles according to the invention in a perfume are, for example, natural products such as extracts, essential oils, absolutes, resinoids, resins, concretes etc., but also synthetic materials such as hydrocarbons, alcohols, aldehydes, ketones, ethers, acids, esters, acetals, ketals, nitriles, etc., including saturated and unsaturated compounds, aliphatic, carbocyclic, and heterocyclic compounds.

Such fragrance materials are mentioned, for example, in S. Arctander, Perfume and Flavor Chemicals (Montclair, N.J., 1969), in S. Arctander, Perfume and Flavor Materials of Natural Origin (Elizabeth, N.J., 1960) and in “Flavor and Fragrance Materials—1991”, Allured Publishing Co. Wheaton, Ill. USA.

Examples of fragrance materials which can be used in combination with one or more nitriles according to the invention are: geraniol, geranyl acetate, linalol, linalyl acetate, tetrahydrolinalol, citronellol, citronellyl acetate, dihydromyrcenol, dihydromyrcenyl acetate, tetrahydromyrcenol, terpineol, terpinyl acetate, nonpol, nopyl acetate, 2-phenyl-ethanol, 2-penylethyl acetate, benzyl alcohol, benzyl acetate, benzyl salicylate, styrallyl acetate, benzyl benzoate, amyl salicylate, dimethylbenzyl-carbinol, trichloromethylphenyl-carbinyl acetate, p-tert-butylcyclohexyl acetate, isononyl acetate, vetiveryl acetate, vetiverol α-hexylcinnamaldehyde, 2-methyl-3-(p-tert-butylpheyl)propanal, 2methyl-3-(p-isopropylphenyl)propanal, 2-(p-tert-butylpheyl)-propanal, 2,4-dimethyl-cyclohex-3-enyl-carboxaldehyde, tricyclodecenyl acetate, tricyclodecenyl propionate,4-(4-hydroxy-4-methylpentyl)-3-cyclohexenecarboxyaldehyde, 4-(4-methyl-3-pentenyl)-3-cyclohexenecarboxaldehyde, 4-acetoxy-3-pentyl-tetrahydropyran, 3-carboxymethyl-2-pentylcyclopentane, 2-n-heptylcyclopentanone, 3-methyl-2-pentyl-2-cyclopentenone, n-decanal, n-dodecanal, 9-decenol-1, phenoxyethyl isobutyrate, phenyl-acetaldehyde dimethylacetal, phenylacetaldehyde diethylacetal, geranyl nitrile, citronellyl nitrile, cedryl acetate, 3-isocamphylcyclohexanol, cedryl methyl ether, isolongifolanone, aubepine nitrile, aubepine, heliotropin, coumarin, eugenol, vanillin, diphenyl oxide, hydroxycitronellal, ionones, methylionones, isomethylionones, irones, cis-3-hexenol and esters thereof, indan musks, tetralin musks, isochroman musks, macrocyclic ketones, macrolactone musks, ethylene brassylate.

Solvents which can be used for perfumes which contain a nitrile according to the invention are, for example: ethanol, isopropanol, diethyleneglycol monoethyl ether, dipropylene glycol, diethyl phthalate, triethyl citrate, isopropyl myristate, etc.

The quantities in which one or more nitriles according to the invention can be used in perfumes or in products to be perfumed may vary within wide limits and depend, inter alia, on the nature of the product, on the nature and the quantity of the other components of the perfume in which the nitrile is used and on the olfactive effect desired. It is therefore only possible to specify wide limits, which, however, provide sufficient information for the specialist in the art to be able to use a nitriles according to the invention for his specific purpose. In perfumes an amount of 0.01% by weight or more of a nitrile according to the invention will generally have a clearly perceptible olfactive effect. Preferably the amount is 0.1 to 80% by weight, more preferably at least 1%. The amount of the nitrile according to the invention present in products will generally be at least 10 ppm by weight, preferably at least 100 ppm, more preferably at least 1000 ppm. However, levels of up to about 20% by weight may be used in particular cases, depending on the product to be perfumed.

The nitriles exist in various isomeric forms. The invention covers each isomeric form alone, and mixtures of different isomeric forms, and also the use in perfumes and perfumed products of separate isomers and mixtures of isomers.

The currently preferred nitriles in accordance with the invention have R═CH₃ and R¹═H. In this case nitrile 1 has the structure 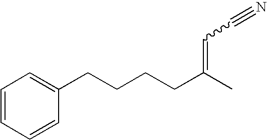
and is 3-methyl-7-phenyl-2-heptenenitrile; nitrile 2 has the structure 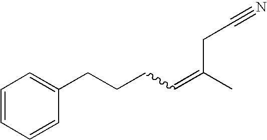
and is 3-methyl-7-phenyl-3-heptenenitrile. Both of these materials exist as both E and Z isomers. Both of these preferred nitriles have peach, fruity, lactone, nitrile odour properties. The nitrile aspects of the odour properties also give the materials power and intensity of smell. Both of these materials are very substantive on both wet and dry cloth, and so have good potential for use in fabric treatment products such as fabric detergents.

In a further aspect the invention provides a perfume comprising one or more nitriles of the invention in an olfactively effective amount.

The invention also covers a perfumed product comprising one or more nitriles of the invention.

Nitriles in accordance with the invention may be synthesised by Aldol condensation of cinnamaldehyde and a suitable ketone, followed by hydrogenation. A Wadsworth Emmons reaction may be performed on the product, to yield a version 1 nitrile. Alternatively, a Knoevenagel condensation may be performed on the product, to yield a mixture of version 1 and version 2 nitriles, which may be used as a mixture or separated. For example, the preferred nitriles referred to above, 3-methyl-7-phenyl-2-heptenenitrile and 3-methyl-7-phenyl-3-heptenenitrile, may be readily and cheaply synthesised by Aldol condensation of cinnamaldehyde with acetone, followed by hydrogenation to yield 6-phenylhexan-2-one. Wadsworth Emmons reaction of 6-phenylhexan-2-one yields 3-methyl-7-phenyl-2-heptenenitrile (as a mixture of E and Z isomers). Alternatively, Knoevenagel condensation of 6-phenylhexan-2-one with cyanoacetic acid yields a mixture of 3-methyl-7-phenyl-2-heptenenitrile and 3-methyl-7-phenyl-3-heptenenitrile (each as a mixture of E and Z isomers). Reaction of the mixture with potassium tert-butoxide yields 3-methyl-7-phenyl-2-heptenenitrile.

In a further aspect, the invention thus provides a method of making a nitrile in accordance with the invention, comprising
i) performing an Aldol condensation of cinnamaldehyde with a ketone;
ii) hydrogenating the product of step i); and
iii) performing a Wadsworth Emmons reaction or a Knoevenagel condensation reaction on the product of step ii).

6-phenylhexan-2-one, which has the following structure: 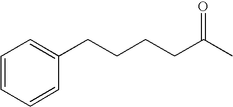
which is produced as an intermediate in the process referred to above, has jasmine-like odour properties, and so has potential fragrance use as an inexpensive jasmine-type filler material. While this material is known per se in the literature, the odour properties of the material have not previously been disclosed. The present invention thus also includes within its scope fragrance uses of this material.

Thus, the invention also provides a perfume comprising 6-phenylhexan-2-one in an olfactively effective amount.

In a further aspect, the invention also covers a perfumed product comprising 6-phenylhexan-2-one.

The invention, in a further aspect, also covers a process for imparting a jasmine-type odour note to a perfume or perfumed product, comprising incorporating 6-phenylhexan-2-one into the perfume or product.

The terms perfume and perfumed product as used here have the same meaning as discussed above, and should be construed accordingly.

The invention will be further described, by way of illustration, in the following Examples.

### EXAMPLE 1

This example describes synthesis routes for 3-methyl-7-phenyl-2-heptenenitrile (material III in the reaction scheme below), and a mixture of 3-methyl-7-phenyl-3-heptenenitrile (material IV in the reaction scheme below) with 3-methyl-7-phenyl-2-heptenenitrile, involving production as an intermediate 6-phenylhexan-2-one (material II in the reaction scheme below).

The synthesis routes are illustrated in the following reaction scheme:

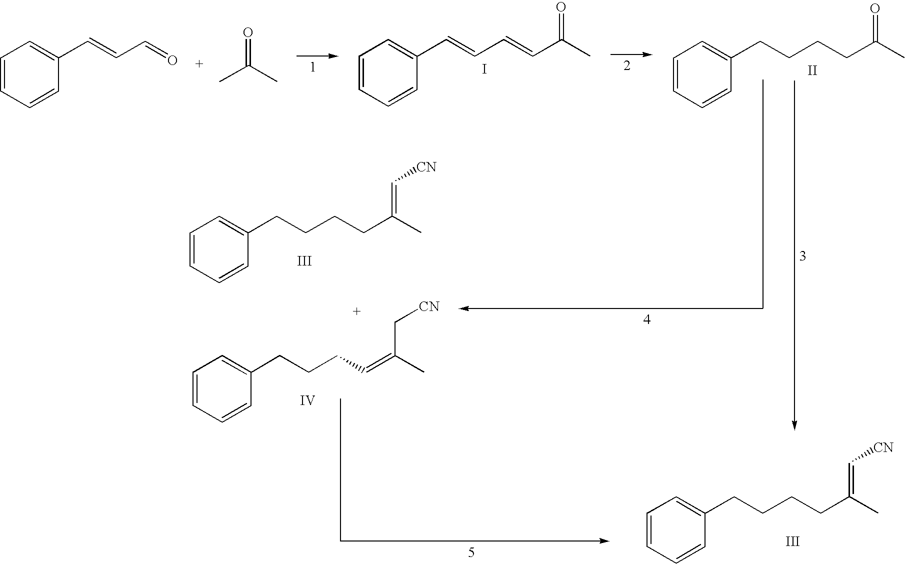

Reaction 1 Aldol Reaction Between Cinamaldehyde and Acetone to Produce I

| | |
|---|---|
| Cinamaldehyde | 396.0 g, 3.0 mol |
| Acetone | 660 ml, 9.0 mol |
| Sodium Hydroxide | 60.0 g, 1.5 mol |
| Water | 6 ltr |

The above four ingredients were heated to 70° C. (using an immersion circulator) and then allowed to cool. A yellow solid precipitated/crystallised out on cooling. The solid was dissolved by the addition of ethyl acetate with stirring. The organic solution was separated, dried with magnesium sulphate, and concentrated down to approximately 1200 ml (91% of one component by GC). This solution was used in the next step without any further purification.

Reaction 2 Hydrogenation of Crude Aldol Product to Yield II

| | |
|---|---|
| Crude Aldol product | 1200 ml |
| solution from reaction 1 | |
| Palladium (5% on carbon) | 2 × 5 g (Hydrogenated in two portions) |
| Hydrogen | |

The solution was hydrogenated in two portions in a glass Buchi autoclave at 4-6 bar. Initially each portion showed an exotherm where the temperature rose up to 45-50° C. The reaction was monitored by GC and once complete was filtered through celite and the residual solvent removed by evaporation. The resulting residue was distilled to yield the following two fractions which were both found to be the desired product:

87-88° C. @ 0.1 mmHg, 132.9 g (97.7% by GC)

90-110° C. @ 0.2 mmHg, 277.0 g (97.6% by GC)

¹³C NMR data analysis: (CDCl₃, ref 77.0 ppm) 208.9 (C═O), 142.3 (CQ), 128.5 (CH), 128.4 (CH), 125.8 (CH), 43.6 (CH), 35.8 (CH₂), 31.0(CH₂), 29.9 (CH₃), 23.5 (CH₂)

Preparation of Target Molecule III

Route 1:

Reaction 3 Wadsworth Emmons Horner Reaction on II

| | |
|---|---|
| 6-Phenylhexan-2-one (II) | 133.0 g, 0.76 mol |
| n-BuLi (10M, Aldrich, in THF) | 88 ml, 0.88 mol |
| Tetrahydrofuran (HPLC grade) | 1 ltr |
| Diethylcyanomethylphosphonate | 141.0 g, 0.80 mol |

The n-BuLi solution (pyrophoric) was added dropwise to the phosphonate in tetrahydrofuran over a period of 1-2 hrs whilst maintaining the temperature below 50° C. (this addition is exothermic). The reaction mixture became orange/clear in appearance with a small amount of colourless solid also being present. This solution was left overnight.

To this orange solution was then added the ketone 6-phenylhexan-2-one over a period of about one hour. This addition was mildly exothermic with the temperature rising to 45° C. over the course of the addition. Once the addition was complete the resulting solution was left to cool overnight. The solvent was removed under reduced pressure and the residue was poured into water (1 ltr). This was then extracted with diethyl ether (2×500 ml) and the combined organic fractions were then washed with water (500 ml), dried with magnesium sulphate, and evaporated under reduced pressure. The resulting residue (148 g) was distilled under reduced pressure to yield a colourless liquid (B.p. 140-142° C. @ 0.5 mmHg, 104.5 g), comprising a mixture of E and Z isomers of 3-methyl-7-phenyl-2-heptenenitrile which displays a peach, fruity, lactone, nitrile odour.

¹³C NMR data analysis: (CDCl₃, ref 77.0 ppm) 117.2/4 (CN), 95.3/9 (CH), 165.4/5 (CQ), 21.0/22.8 (CH₃), 38.6 (CH₂), 26.7/27.1 (CH₂), 30.9 (CH₂), 36.1/35.7 (CH₂), 142.1/2 (CQ), 128.5 (CH), 128.6 (CH), 126 (CH)

Route 2:

Reaction 4 II Plus Cyanoacetic Acid (Knoevenagel Condensation)

| | |
|---|---|
| 6-Phenylhexan-2-one (II) | 176.0 g, 0.10 mol |
| Cyanoacetic acid | 93.5 g, 0.11 mol |
| Piperidine | 50 ml |
| Benzene | 1 ltr |

Piperidine was added to 6-phenylhexan-2-one plus cyanoacetic acid in benzene. The temperature rose from 20° C. to 35° C. and the reaction mixture temporarily solidified before turning liquid again. The reaction mixture was heated at reflux in Dean and Stark apparatus until the theoretical amount of water had been removed.

The reaction mixture was washed twice with water (2×500 ml) and then the solvent was removed under reduced pressure. The residue was distilled to give a mixture of III and IV (each as a mixture of E and Z isomers) as a colourless liquid (B.p. 124-128° C. @ 0.1 mmHg, 171.3 g), which displays a peach, lactone, fruity, nitrile odour.

¹³C NMR data analysis: Compound III (CDCl₃, ref 77.0 ppm) 117.2/4 (CN), 95.3/9 (CH), 165.4/5 (CQ), 21.0/22.8 (CH₃), 38.6 (CH₂), 26.7/27.1 (CH₂), 30.9 (CH₂), 36.1/35.7 (CH₂), 142.1/2 (CQ), 128.5 (CH), 128.6 (CH), 126 (CH) Compound IV (CDCl₃, ref 77.0 ppm) 118.0 (CN), 27.7 (CH₂), 130.0 (CQ), 16.2 (CH₃), 124.5 (CH), 27.4 (CH₂), 36.0 (CH₂), 142.1 (CQ), 128.5 (CH), 128.6 (CH), 125.9 (CH)

Reaction 5 Reaction of III+IV With Base

| | |
|---|---|
| Mixture of III + IV (as obtained | 137.9 g, 0.69 mol |
| from distillation in reaction 4) | |
| Potassium tert-butoxide | 14.0 g, 0.13 mol |
| Cyclohexane | 500 ml |

To a mixture of III/IV (as obtained from distillation in reaction 4) in cyclohexane with stirring was added potassium tert-butoxide in one portion. The temperature rose from 20° C. to 35° C. and the colour changed from pale yellow to dark brown almost immediately. After 5 minutes the reaction was washed with water (3×500 ml), dried with anhydrous magnesium sulphate, and evaporated under reduced pressure to yield a colourless product. The product was distilled to give a colourless liquid (III) (B.p. 140-142° C. @ 0.5 mmHg, 131.2 g).

¹³C NMR data analysis: (CDCl₃, ref 77.0 ppm) 117.2/4 (CN), 95.3/9 (CH), 165.4/5 (CQ), 21.0/22.8 (CH₃), 38.6 (CH₂), 26.7/27.1 (CH₂), 30.9 (CH₂), 36.1/35.7 (CH₂), 142.1/2 (CQ), 128.5 (CH), 128.6 (CH), 126 (CH)

GC analysis:

| | | | | | | Mixed | |
|---|---|---|---|---|---|---|---|
| Reaction 3 | | Reaction 4 | | Reaction 5 | | GC | |
| Product Isomer | | Product Isomer | | Product Isomer | | Routes | |
| Ratio | | Ratio | | Ratio | | 3, 4 & 5 | Isomer |
| Minutes | Crude % | Minutes | Crude % | Minutes | Crude % | Minutes | Assignment |
| — | — | 6.47 | 4 | — | — | 6.48 | Z-IV |
| 6.64 | 36 | 6.62 | 6 | 6.63 | 34 | 6.64 | Z-III |
| — | — | 6.80 | 50 | — | — | 6.81 | E-IV |
| 6.97 | 57 | 6.95 | 39 | 6.95 | 63 | 6.98 | E-III |

### EXAMPLE 2

A perfume in accordance with the invention was prepared by mixing together the following materials:

| | % by weight |
|---|---|
| Bangalol (Quest) | 5 |
| Hexyl cinnamic aldehyde | 12 |
| Ionone beta | 5 |
| Iso bornyl cyclo hexanol | 5 |
| Lily aldehyde | 6 |
| Mefrosol (Quest) | 18 |
| Methyl dihydro jasmonate super (Quest) | 8 |
| Nectaryl (givaudan) | 2 |
| Peach Nitrile (Nitrile 1 and/or 2) | 4 |
| Ortholate (Quest) | 24 |
| Ethyl Safranate (Quest) | 1 |
| Galaxolide pure | 10 |

Bangalol, Mefrosol, Methyl dihydro jasmonate super, Nectaryl, Ortholate and Ethyl Safranate are all Trade Marks.

1. A compound having the structure 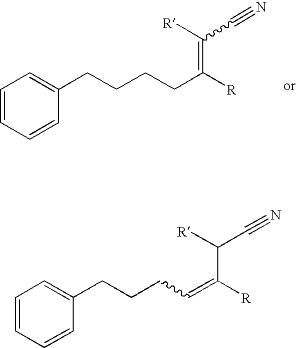
where R and R¹ is each independently hydrogen or an alkyl, alkenyl, cycloalkyl or cycloalkenyl group having from 1 to 5 carbon atoms.

2. A compound according to claim 1, having the structure

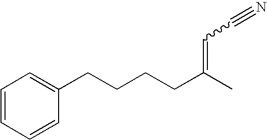

3. A compound according to claim 1, having the structure

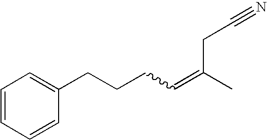

4. A perfume comprising one or more compounds in accordance with any one of the preceding claims in an olfactively effective amount.

5. A perfume according to claim 4, wherein the compound is present in an amount of at least 0.01% by weight.

6. A perfume according to claim 5, wherein the compound is present in an amount in the range 0.1 to 80% by weight.

7. A perfumed product comprising one or more compounds according to claim 1, 2 or 3 or a perfume according to claim 4, 5 or 6.

8. A method of making a compound in accordance with claim 1, comprising
i) performing an Aldol condensation of cinnamaldehyde with a ketone;
ii) hydrogenating the product of step i); and
iii) performing a Wadsworth Emmons reaction or a Knoevenagel condensation reaction on the product step ii).

9. A perfume comprising 6-phenylhexan-2-one in an olfactively effective amount.

10. A perfumed product comprising 6-phenylhexan-2-one.

11. A process for imparting a jasmine-type odour note to a perfume or performed product, comprising incorporating 6-phenylhexan-2-one into the perfume or perfumed product.
